(19) United States
(12) Patent Application Publication
(10) Pub. No.: US 2021/0379221 A1
(43) Pub. Date: Dec. 9, 2021

(54) SYSTEM AND METHODS FOR GAMING DEVICES HAVING STERILIZATION FEATURES

(71) Applicant: SHORTWAVE GAMING TECHNOLOGIES, LLC, Dix Hills, NY (US)

(72) Inventors: David Ridell, Dix Hills, NY (US); Marc Ridell, Dix Hills, NY (US)

(21) Appl. No.: 17/340,876

(22) Filed: Jun. 7, 2021

Related U.S. Application Data
(60) Provisional application No. 63/036,171, filed on Jun. 8, 2020.

(51) Int. Cl. A61L 2/10; A61L 2/24; A63F 1/12; A63F 1/06

(52) U.S. Cl. CPC A61L 2/10; A61L 2/24; A61L 2202/14; A63F 1/062; A61L 2202/11; A63F 1/12

(57) ABSTRACT

The present disclosure includes, systems, devices, and methods for sterilizing objects, such as gaming equipment or gaming pieces. Some aspects include a sterilization device configured to be integrate with, or coupled to, gaming equipment to sterilize a plurality of gaming pieces. The device may include a housing defining a chamber configured to receive a plurality of gaming pieces and one or more light source(s) coupled to the housing and configured to emit ultraviolet (UV) light toward the gaming pieces.

### CROSS-REFERENCE TO RELATED APPLICATIONS

This application claims priority to U.S. Provisional Application No. 63/036,171, filed Jun. 8, 2020, the contents of which is incorporated herein by reference in its entirety.

### FIELD OF INVENTION

The present invention relates generally to gaming systems with integrated sterilization systems, and more specifically, to methods and systems for UV sterilization of gaming equipment in a casino gaming environment such as cards, dice, chips, cash, card shufflers, drop-boxes, chip sorters, chip storage trays, dealer shoes, discard trays, cash registers, or the like.

### BACKGROUND

The likelihood of passing germs, bacteria, viruses or other dangerous pathogens has been traditionally problematic in forums where items are passed as a matter of course between the public and employees. The problem increases when the forum includes fixed equipment that is shared by individuals. For example, in casinos, chips, dice, cards, cash and other items continuously pass between countless individuals. Moreover, in casinos, countless individuals use the same playing card tables, slot machines, video poker machines, and/or other games of chance. In many cases, these items are infrequently sterilized and interruptions in use for sterilization are undesirable at least because many casinos operate on a 24-hour basis and because such gaming devices are under intense supervision and security.

While issues regarding sanitation may affect any public forum where items are passed between the individuals or where individuals share the use of equipment, casinos, unlike other public forums, are particularly susceptible to the transmission of germs, bacteria, viruses or other dangerous pathogens because the casino environment typically includes crowds of people from all over the world who, for many hours a day, are drinking and/or smoking (each an activity that transfers pathogens from hand to mouth) while they gamble and socialize.

Traditionally, for casinos that do employ measures for sterilizing chips, dice, cards, cash and other items, such sterilization is done in an independent, dedicated sterilization machine that is either (a) stored and used in a secure portion of the casino, away from the players, or (b) on the casino's gaming floor, taking up valuable space that could otherwise be occupied by paying patrons. Additionally, such sterilization typically involves the use of harsh chemical solutions that can take several minutes or hours to properly disinfect.

Accordingly, there is a need to provide a convenient, non-obtrusive, and cost-effective solution to improve the sterilization of gaming pieces to thereby reduce the transmission of dangerous germs, bacteria, viruses, and other dangerous pathogens between individuals in casinos.

### SUMMARY

The present disclosure relates to the incorporation of UV sterilization features within or in conjunction with gaming equipment, such as existing card shufflers, drop-boxes, chip sorters, chip storage trays, dealer shoes, discard trays, cash registers, or the like. Some aspects of present disclosure include, systems, devices, and methods for sterilizing objects, such as gaming equipment or gaming pieces. Some aspects include a sterilization device configured to be integrate with, or coupled to, gaming equipment to sterilize a plurality of gaming pieces. The device may include a housing defining a chamber configured to receive a plurality of gaming pieces and one or more light source(s) coupled to the housing and configured to emit ultraviolet (UV) light toward the gaming pieces. The light source(s) are configured or arranged to direct the emitted UV light toward the gaming pieces at an energy sufficient to sterilize the gaming pieces without damaging the gaming pieces.

In some aspects, the housing can be coupled to a card playing table. In some embodiments, the device corresponds to a card shuffler, a chip sorter, or a cash drop box. As a non-limiting example, the housing may define a first opening configured to receive the gaming pieces and a second opening configured to dispense the gaming pieces. In some such embodiments, the light source(s) are positioned such that the gaming pieces are sterilized as they pass through the first opening or the second opening. Additionally, or alternatively, the housing may include a platform disposed within the chamber. In some such embodiments, at least one of the light sources is disposed on a first side of the platform and a reflector or one other light source is disposed on a second side of the platform, the second side being opposite the first side.

Some embodiments of the present devices include a sorting system configured to sort the plurality of gaming pieces. The sorting system may be integrated with, or coupled to, the housing. In some aspects, the light sources are configured to emit pulsed UV light. Some such embodiments include a controller in communication with the light source. The controller may be configured to identify a type of the plurality of gaming pieces. In some such embodiments, based on the identified type, the controller may be configured to select a number of pulses to be delivered to the plurality of gaming pieces.

Some aspects of the present devices may include a housing configured to be coupled to gaming equipment and one or more ultraviolet (UV) light sources coupled to the housing. In some aspects, the housing may have an opening configured to transfer gaming pieces between the housing and the gaming equipment. In some embodiments, the UV light sources coupled to the housing and positioned to deliver UV light to the gaming pieces. As a non-limiting example, the UV light sources may be configured to emit pulsed UV light such that the gaming pieces are subject to at least 2 log reduction. In some embodiments, at least one of the one or more ultraviolet (UV) light sources are positioned within the housing. In some aspects, the gaming equipment may correspond to a card shuffler or a chip sorter.

In some aspects, the housing is coupled to the gaming equipment such that the gaming pieces are received by the housing from the gaming equipment. In other aspects, the housing is coupled to the gaming equipment such that the gaming pieces are delivered to the gaming equipment from the housing. The gaming pieces may include chips, die, cards, or cash.

Some aspects of the present methods include a method for sanitizing gaming pieces. Some such methods include applying ultraviolet (UV) light, via a gaming device having one or more UV light sources, to a plurality of gaming pieces to sterilize the gaming pieces. In some aspects, the gaming device may be coupled to a card playing table. In some methods, applying ultraviolet light includes applying five or more light pulses. In some such methods, each light pulse having an energy greater than 12 joules per pulse. In some methods, the UV light includes a wavelength that is between 100 and 400 nanometers. Some of the present methods may further include disposing the gaming pieces within a housing of the gaming device. Some methods may include sorting the gaming pieces. Some such methods may include dispensing the gaming pieces from the housing after the gaming pieces are sorted and sterilized.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The terms “a” and “an” are defined as one or more unless this disclosure explicitly requires otherwise.

The term “about” as used herein can allow for a degree of variability in a value or range, for example, within 10%, within 5%, or within 1% of a stated value or of a stated limit of a range, and includes the exact stated value or range. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed implementation, the term “substantially” may be substituted with “within [a percentage] of” what is specified, where the percentage includes 0.1, 1, or 5 percent; and the term “approximately” may be substituted with “within 10 percent of” what is specified. The statement “substantially X to Y” has the same meaning as “substantially X to substantially Y,” unless indicated otherwise. Likewise, the statement “substantially X, Y, or substantially Z” has the same meaning as “substantially X, substantially Y, or substantially Z,” unless indicated otherwise. Unless stated otherwise, the word or as used herein is an inclusive or and is interchangeable with “and/or.” To illustrate, A, B, or C includes: A alone, B alone, C alone, a combination of A and B, a combination of A and C, a combination of B and C, or a combination of A, B, and C. Similarly, the phrase “A, B, C, or a combination thereof” or “A, B, C, or any combination thereof” includes: A alone, B alone, C alone, a combination of A and B, a combination of A and C, a combination of B and C, or a combination of A, B, and C.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any implementation of any of the systems, methods, and article of manufacture can consist of or consist essentially of—rather than comprise/have/include—any of the described steps, elements, or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, the term “wherein” may be used interchangeably with “where”.

Further, a device or system that is configured in a certain way is configured in at least that way, but it can also be configured in other ways than those specifically described. The feature or features of one implementation may be applied to other implementations, even though not described or illustrated, unless expressly prohibited by this disclosure or the nature of the implementations.

Some details associated with the implementations are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configuration depicted in the figures.

FIG. 1 shows a card playing table and a first embodiment of the present gaming devices having UV sterilization features.

FIG. 2 shows a schematic of the first embodiment of the present gaming devices.

FIG. 3 shows a schematic of a second embodiment of the present gaming devices having UV sterilization features.

FIG. 4 shows a schematic of a third embodiment of the present gaming devices having UV sterilization features.

FIG. 5 shows a schematic of a fourth embodiment of the present gaming devices having UV sterilization features.

FIG. 6A shows a table and a fifth embodiment of the present gaming devices having UV sterilization features.

FIG. 6B shows a schematic of the fifth embodiment of the present gaming devices.

FIG. 7A shows a side view of an experimental setup of a UV light source and a testing sample.

FIG. 7B shows a graph depicting the relationship between energy density applied to the testing sample and a distance from the UV light source of the experimental setup of FIG. 7A.

FIG. 8 shows two die that were exposed to UV light using the experimental setup of FIG. 7A.

### DETAILED DESCRIPTION

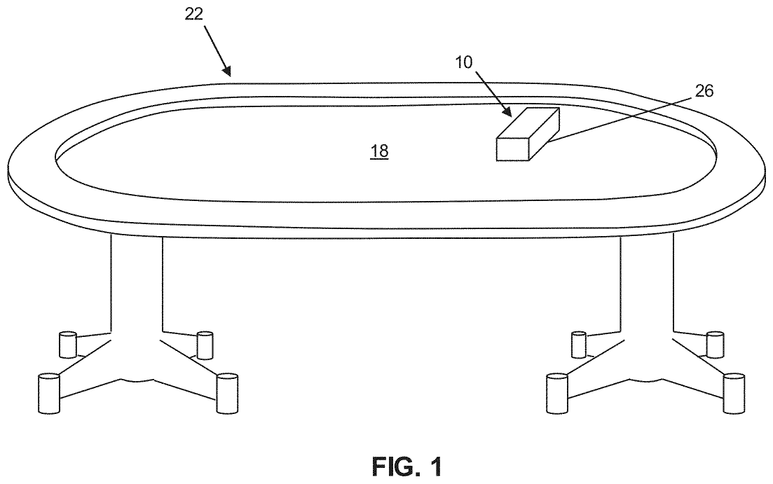

Referring to FIG. 1, an illustrative depiction of one embodiment of the present gaming devices having sterilization features is shown and generally designated by reference numeral 10. As shown, device 10 includes a housing 14 configured to be disposed on a surface 18 of a card playing table 22, such as, for example, a poker table, a blackjack table, and/or the like.

As described in further detail herein, housing 14 can include one or more features for identifying, organizing, storing, and/or shuffling gaming pieces, such as playing cards, dice, chips, cash, or the like. Device 10 may be configured to extend at least partially above and/or below a playing surface 18 of table 22. More particularly, in embodiments where device 10 extends at least partially below playing surface 18, table 22 can comprise an opening 26 within which the device is disposed (e.g., drop box). In some embodiments, a device (e.g., 10) is positioned on top of a playing surface (e.g., 18) of a table (e.g., 22), and thus an opening (e.g., 26) in the table may be omitted. Device 10 can be any suitable size such that it is easily accessible to a dealer during the ordinary course of play and such that it does not obstruct the dealer's or a player's ordinary physical movement during the course of play.

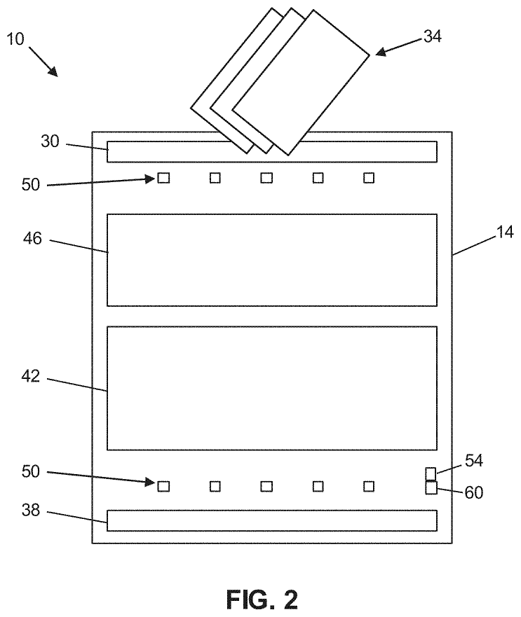

Referring additionally to FIG. 2, housing 14 may comprise a feeder 30 configured to receive a plurality of gaming pieces 34, such as cards (e.g., individually and/or more than one at a time) and a dispenser 38 configured to permit removal of the gaming pieces (e.g., individually and/or more than one at a time) from the housing. Feeder 30 and/or dispenser 38 of shuffler housing 14 can each or collectively define an opening in the housing, a tray coupled to the housing, a recessed portion of the housing, and/or the like, as is known in the art.

In the depicted embodiment, device 10 may include a shuffling system 42 configured to shuffle gaming pieces 34 within shuffler housing 14. For example, shuffling system 42 can be configured to receive cards (e.g., 34) that have been inserted into feeder 30, shuffle the cards, and then directly or indirectly supply the shuffled cards to dispenser 38. Such shuffling system 42 can comprise any suitable mechanical, electronic, optical, RFID, and/or the like features, as is known in the art, for shuffling cards within shuffler housing 14. Suitable examples of a shuffling system (e.g., 42) that can be used with device 10 are disclosed in U.S. Pat. Nos. 3,944,230, 4,807,884, and 10,576,363, the contents of which are hereby incorporated by reference in their entirety.

Device 10 can comprise a sorting system 46 configured to identify and organize gaming pieces 34 (e.g., cards) within housing 14. For illustrative purposes, sorting system 46 is described in operation with playing cards. Sorting system 46 may be in series between feeder 30 and shuffling system 42 (i.e., the sorting system identifies and/or sorts cards before the cards are shuffled by the shuffling system). For illustrative purposes, sorting system 46 can be configured to receive cards (e.g., 34) that have been inserted into feeder 30, identify the cards on an individual basis, store the cards according to an organization scheme, and then directly or indirectly supply the cards to shuffling system 42. By identifying, organizing, and storing cards (e.g., 34), sorting system 46 can facilitate more effective shuffling by shuffling system 42. Sorting system 46 can comprise any suitable mechanical, electronic, optical, RFID, and/or the like features, as is known in the art, for identifying, organizing, and storing cards within shuffler housing 14. Suitable examples of a sorting system (e.g., 46) that can be used with device 10 are disclosed in U.S. Pat. No. 6,250,632 and U.S. Pub. No. 2002/0017481, the contents of which are hereby incorporated by reference in their entirety. In some embodiments, the components of a sorting system (e.g., 46) are included in a shuffling system (e.g., 42), and as such an independent sorting system (e.g., 46) can be omitted. In yet other embodiments, sorting system 46 may be configured to receive other gaming pieces, such as chips, and sort the gaming pieces based on size, color, weight, images, or other distinguishing features.

Device 10 can have any suitable mechanical and/or electronic features, as is known in the art, to move gaming pieces 34 (e.g., cards) between one or more components of the device, such as feeder 30, sorting system 46, shuffling system 42, and dispenser 38, and such movement may be gravity-assisted.

As shown in FIG. 2, device 10 comprises one or more ultraviolet (UV) light sources 50 configured to sterilize the gaming pieces 34 by exposing the pieces to suitable wavelengths of UV light. More particularly, UV light source(s) 50 can include a UV illumination source coupled to a power supply (e.g., an independent power supply, an existing power supply within shuffler housing 14, or both) such that the light source is configured to generate light between 100 and 400 nanometers wavelength (e.g., at least one of UV-A, UV-B, or UV-C). At least one of UV light source(s) 50 can comprise a conventional bulb, filament, LED, and/or the like and may be configured to emit light continuously, in one or more patterns (e.g., pulsed, strobed, or the like), or both. For example, UV light source(s) 50 may be configured to operate at a pulse rate between 3 and 100 pulses per second (pps) or at a duty cycle between 5% and 100%. In some embodiments, at least one of UV light source(s) 50 can comprise one or more suitable light diffusion panels configured to scatter the emitted UV light. Device 10 may also include one or more sensors and/or timers 54 and related controllers 60 configured to control the duration, number of pulses, pulse rate, energy or other characteristics of UV light exposure to cards (e.g., 34), in order to ensure proper sterilization.

As used herein, the terms “sterilization,” “sterilize,” or any other variation thereof includes any process or technique directed to sanitizing, cleaning, making hygienic, disinfecting, and/or otherwise making free from bacteria or other living microorganisms, in whole or in part. In some embodiments, sterilization may be measured using sterility assurance level (SAL), log reduction, or the like. For example, some methods of sterilizing gaming pieces 34 (e.g., using UV light) may have a SAL that is greater than, equal to, or between any two of 10⁻¹, 10⁻², 10⁻³, 10⁻⁴, 10⁻⁵, or 10⁻⁶ Additionally, or alternatively, some of the described methods of sterilization may have a 1, 2, 3, 4, 5, 6, 7, 8, 9, 10, 11, 12 log reduction, or greater. Accordingly, “sterilization,” or “sterilize,” as used herein, may refer to applying UV light to an object to achieve a SAL greater than 10¹ (e.g., between 10⁻² and 10⁻¹²) or achieve a 1 log reduction or greater (e.g., between 2 and 12 log reduction).

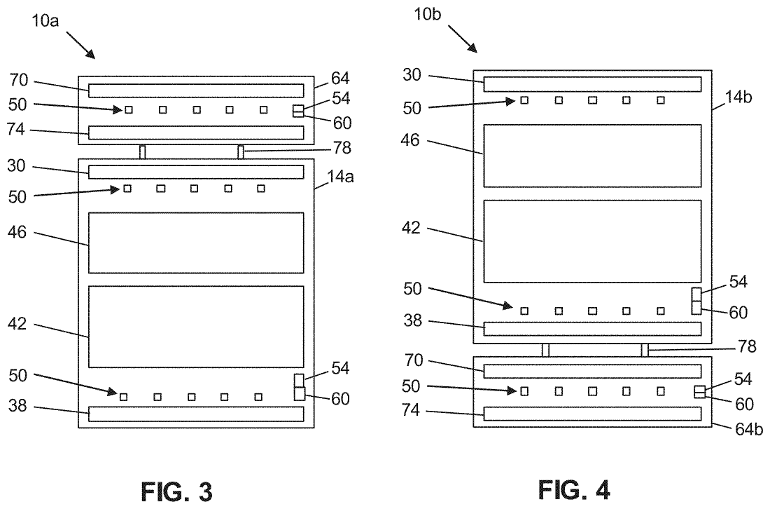

UV light source(s) 50 can be positioned at any suitable location relative to shuffler housing 14 in order to sterilize cards (e.g., 34). To illustrate, in some embodiments, such as the embodiment shown in FIG. 2, a device (e.g., 10) can include a shuffler housing (e.g., 14) having at least one UV light source (e.g., 50). To further illustrate, in some embodiments, such as the embodiments shown in FIGS. 3-5, a device (e.g., 10a, 10b, 10c) can include at least one UV light source (e.g., 50) that is not incorporated into a shuffler housing (e.g., 14a, 14b, 14c) and is instead included in an independent UV housing (e.g., 64, 64b, 64c) outside of the shuffler housing.

As shown in the embodiment depicted in FIG. 2, device 10 can comprise one or more UV light source(s) 50 within housing 14 and proximate to and/or at feeder 30 such that, as cards, or other gaming pieces 34, are placed into the feeder (or shortly thereafter), the cards are exposed to UV light and thereby sterilized. To further illustrate, device 10 can comprise one or more UV light source(s) 50 within shuffler housing 14 and proximate to and/or at dispenser 38 such that, shortly before (or as) cards, or other gaming pieces 34, are withdrawn from shuffler housing 14 via the dispenser, the cards are exposed to UV light and thereby sterilized. In some embodiments, a device (e.g., 10) can comprise one or more UV light source(s) (e.g., 50) incorporated into a shuffling system (e.g., 42) and/or a sorting system (e.g., 46) and/or between such shuffling systems and such sorting systems to sterilize the cards within a housing (e.g., 10). In some embodiments, at least one of UV light source(s) (e.g., 50) can be disposed on an exterior surface of a housing (e.g., 14) such that cards can be passed over the UV light source(s) to sterilize the cards.

One or more of UV light source(s) 50 that are disposed within shuffler housing 14 can, for example, be positioned such that the UV light source (and the light emitted therefrom) is blocked from exposure to a user during ordinary use of device 10. On the other hand, one or more of UV light source(s) 50 that are disposed within shuffler housing 14 can, for further example, be positioned such that a portion of the UV light source (and/or the light emitted therefrom) is exposed to a user during ordinary use of device 10. One or more UV light source(s) 50 may include a protective filter or covering to protect a user's eyes and/or skin from harmful UV light exposure, especially in instances where the UV light source(s) and/or the light emitted therefrom are otherwise exposed to the user during ordinary use.

Although the device 10 of FIG. 2 is shown and described as including a card shuffler housing 14, the device may include or correspond to any other gaming equipment. For example, in some embodiments, device 10 and housing 14 may correspond to a chip sorter. In such embodiments, sorting system 46 may be configured to sort chips (e.g., 34), such as poker chips or roulette chips and UV light source(s) 50 are configured to sterilize the chips. The UV light source(s) 50 may be positioned to emit UV light before, during, or after the chips pass through sorting system 46. In some embodiments, UV light source(s) 50 are configured to emit light at higher energies for shorter duration to sterilize gaming pieces 34 without causing excessive delay. For example, the entire sterilization process may be completed (e.g., to a target SAL) within a few minutes, such as, for example less than, equal to, or between any two of: 5, 4, 3, 2, 1 minutes, or 50, 45, 40, 30, 25, 20, 15, or 10 seconds. In some embodiments, the energy of UV light source(s) 50 may be limited to prevent degradation of gaming pieces during the sterilization process. Accordingly, the present devices (e.g., 10) can be arranged or operated, as described herein, to achieve a target sterilization level (e.g., 2 log reduction) of the gaming pieces 34 without damaging the gaming pieces.

Referring additionally to FIG. 3, shown therein and generally designated by reference numeral 10a is an illustrative depiction of another embodiment of the present gaming devices having sterilization features. Device 10a comprises a shuffler housing 14a having features that can be substantially similar to the features of shuffler housing 14, with the primary exceptions described below. In this embodiment, device 10a comprises a UV housing 64 configured to be coupled to shuffler housing 14a. UV housing 64 can comprise one or more UV light source(s) 50 within the UV housing and configured to sterilize cards (e.g., 34) within the UV housing. For example, in this embodiment, UV housing 64 can comprise a feeder 70 configured to receive cards (e.g., 34) and a dispenser 74 configured to dispense cards out of the UV housing after the cards have been sterilized by UV light source(s) 50 within the UV housing. In some embodiments, a UV housing (e.g., 64) can comprise one or more UV light source(s) (e.g., 50) disposed on an exterior surface of the UV housing such that cards can be passed over the UV light source(s) to sterilize the cards.

In the embodiment shown in FIG. 3, UV housing 64 is coupled to shuffler housing 14a such that dispenser 74 of the UV housing is in communication with feeder 30 of the shuffler housing. That is, UV housing 64 can be configured to receive unshuffled (e.g., and pre-sterilized and/or unsterilized) cards into feeder 70, sterilize the cards using UV light source(s) 50 within the UV housing, and dispense sterilized cards to feeder 30 of shuffler housing 14 for shuffling. Device 10a can have any suitable mechanical and/or electronic features, as is known in the art, to effectuate the movement of cards (e.g., 34) from UV housing 64 to shuffler housing 14a as described herein, including features configured to move the cards using air and/or the assistance of gravity.

UV housing 64 can be coupled to a table (e.g., 22) and/or shuffler housing 14a such that at least a portion of the UV housing extends above the playing surface (e.g., 18) of the table. In some embodiments, a UV housing (e.g., 64) can be coupled to a table (e.g., 22) and/or a shuffler housing (e.g., 14a) such that at least a portion of the UV housing extends below the table. UV housing 64 can be coupled to shuffler housing 14a and/or a table (e.g., 22) by any suitable mechanical, electrical, and/or magnetic device 78, such as one or more fasteners, one or more interlocking features, and/or one or more magnets between the UV housing and the shuffler housing, and/or the like.

UV light source(s) 50 within UV housing 64 can be powered by an independent power supply within the UV housing and/or an existing power supply within shuffler housing 14. Similar to shuffler housing 14, UV housing 64 may also include one or more sensors and/or timers 54 and related controllers 60 configured to control the duration of UV light exposure to gaming pieces 34, in order to ensure proper sterilization. Although UV light source(s) 50 are depicted within UV housing 64, in other embodiments, one or more of the UV light source(s) may be positioned external to the housing.

As shown in FIG. 3, shuffler housing 14a can comprise UV light source(s) 50 (in addition to the UV light source(s) within UV housing 64), however such additional UV light sources are optional and, in some embodiments, can be omitted.

To further illustrate, referring additionally to FIG. 4, shown therein and generally designated by reference numeral 10b is an illustrative depiction of another embodiment of the present gaming devices having sterilization features. Device 10b comprises a shuffler housing 14b having features that can be substantially similar to the features of shuffler housing 14 and a UV housing 64b having features that can be substantially similar to the features of UV housing 64, with the primary exceptions described below. In this embodiment, UV housing 64b is configured to be coupled to shuffler housing 14b such that dispenser 38 of the shuffler housing is in communication with feeder 70 of the UV housing 64b. That is, UV housing 64b can be configured to receive shuffled (e.g., and pre-sterilized and/or unsterilized) cards into feeder 70, sterilize the cards using UV light source(s) 50 within the UV housing, and dispense sterilized, shuffled cards via dispenser 74.

Similar to device 10a, device 10b can have any suitable mechanical and/or electronic features, as is known in the art, to effectuate the movement of cards (e.g., 34) from shuffler housing 14b to UV housing 64b as described herein, including features configured to move the cards using air and/or the assistance of gravity. Similar to shuffler housing 14a, shuffler housing 14b can comprise UV light source(s) 50 (in addition to the UV light source(s) within UV housing 64b), however such additional UV light sources are optional and, in some embodiments, can be omitted.

Although the devices 10a and 10b of FIGS. 3 and 4 are shown and described as coupled to a card shuffler housing (e.g., 14a), the devices 10a, 10b may be attachable, or otherwise operational with, any conventional gaming equipment. For example, in some embodiments, devices 10a, 10b may be configured to be coupled to an existing chip sorter, cash sorting machine, other shuffling or storage equipment, or the like. In some embodiments, the present devices (e.g., 10a) may couple to existing gaming equipment to sanitize gaming pieces 34 before they are introduced to the equipment, while in other embodiments, the devices (e.g., 10b) may couple to the existing gaming equipment to sanitize gaming pieces 34 after they leave the equipment. In some embodiments configured to receive gaming pieces 34 from existing gaming equipment, the device (e.g., 10b) may have one or more storage features (e.g., grooves, channels, chambers, or other compartments) to store the gaming pieces 34. The transfer of gaming pieces 34 between the devices (e.g., 10a, 10b) and the equipment may be accomplished with additional mechanical components (e.g., conveyor) or without additional components (e.g., gravity feed).

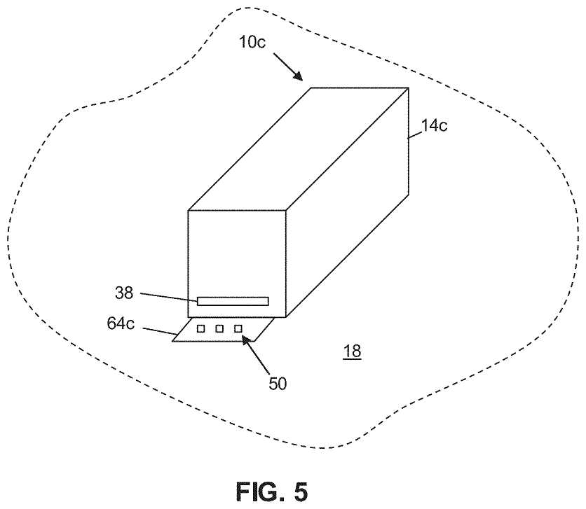

Referring additionally to FIG. 5, shown therein and generally designated by reference numeral 10c is an illustrative depiction of another embodiment of the present gaming devices having sterilization features. Device 10c comprises a shuffler housing 14c having features that can be substantially similar to the features of shuffler housing 14, with the primary exceptions described below. In this embodiment, device 10c comprises a UV housing 64c configured to be coupled to shuffler housing 14c and/or a playing table (e.g., 22). UV housing 64c can comprise one or more UV light source(s) 50 disposed on an exterior surface of the UV housing such that cards (e.g., 34) can be passed over the UV light source(s) to sterilize the cards. To illustrate, cards shuffled by device 10c within shuffler housing 14c can be dispensed via dispenser 38 of the shuffler housing. In this embodiment, such cards may be dispensed onto or over UV light source(s) 50 of UV housing 64c. In some embodiments, such cards pay be dispensed via a dispenser (e.g., 38) and pass over move onto UV light source(s) (e.g., 50) as a user removes the cards from a shuffler housing (e.g., 14c). In order to facilitate movement of dispensed cards over or onto UV light source(s) 50, UV housing 64c may be disposed below dispenser 38 of shuffler housing 14c. As shown, UV housing 64c may be substantially flush with playing surface 18 to allow a smooth transition between the UV housing and the playing surface in instances where a user slides the dispensed cards off of the UV housing. In some embodiments, a UV housing (e.g., 64c) may be slightly above or below a playing surface (e.g., 18) (e.g., less than 5 millimeters between an upper surface of the UV housing and the playing surface) and still allow a relatively smooth transition between the UV housing and the playing surface in instances where a user slides the card off of the UV housing.

The above described devices (e.g., 10, 10a, 10b, 10c), whether incorporated into gaming equipment or coupled to existing gaming equipment, allow for sterilization of gaming pieces without the need to transport the gaming equipment away from the playing table. In this way, players can use uncontaminated gaming pieces without the need for any delays to switch out contaminated gaming pieces for sterilized ones. While the disclosure herein references a housing (e.g., 14, 14a, 14b, 14c) for shuffling cards, the UV sterilization features described herein can be similarly applied to housings for storing used playing cards, or other gaming pieces 34, which are similarly disposed on a playing table (e.g., 22) and which receive spent, typically unsterilized cards. For example, housing may receive cards, or other gaming pieces, at the completion of a card game, while the card game continues, and/or after an allotted number of cards are dealt. Such storage housings can permit the sterilization of cards while the cards are stored within the housing and/or as they are being placed into the housing. Further, the UV sterilization features described herein can be similarly applied to dealer shoes, cash drop boxes (e.g., wherein the UV sterilization features described herein are intended to sterilize cash deposits), and/or the like.

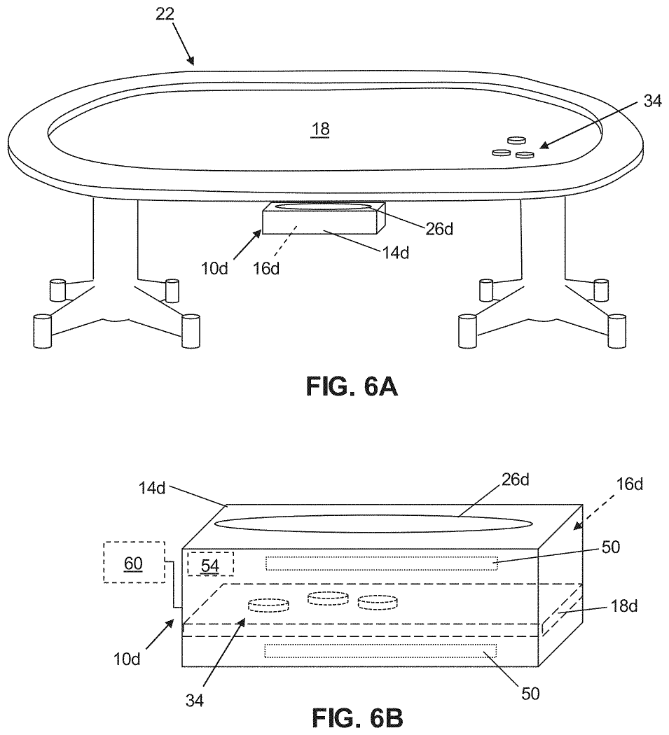

FIG. 6A depicts an illustrative embodiment of a device 10d configured to receive and sterilize gaming pieces 34 such as cash, chips (e.g., poker chips, roulette chips), dice, roulette balls, cards, or any other gaming pieces. As shown, device 10d may be disposed on a table 22, such as a poker table, blackjack table, roulette table, craps table, or any other table configured to be set up on a casino floor. In the depicted embodiment, device 10d is disposed below a table surface 18 of the card playing table (e.g., at a position nearest where a dealer would stand), however, in other embodiments, device 10d may be positioned on top of the table surface or in the same plane as the table surface.

Device 10d includes one or more UV light sources 50 and a housing 14d that defines a chamber 16d and at least one opening 26d in communication with the chamber and configured to receive gaming pieces 34 (e.g., chips). In some embodiments, housing 14d may define multiple openings 26d. For example, housing 14d may define a first opening (e.g., 26d) configured to receive chips (e.g., 34) into chamber 16d and a second opening (e.g., 26d) configured to dispense the chips from the chamber after they have been sterilized. In some embodiments, opening(s) 26d may be selectively closed, or otherwise sealed off, so that access to gaming pieces 34 within housing 14d may be controlled (e.g., for security, convenience, or the like). As an illustrative example, device 10d may include a lid, sliding door, or other means for accessing chamber 16d. Additionally, or alternatively, device 10d may include one or more latches or other locking mechanisms to secure gaming pieces 34 or cash disposed in the chamber 16d.

As shown and described in FIG. 6B, UV light source(s) 50 may be disposed in chamber 16d, or otherwise coupled to housing 14d. In other embodiments, UV light source 50 need not be incorporated into housing 14d and may be external to housing 14d or included in an independent UV housing that is separate from housing 14d. In any such embodiments, UV light source(s) 50 are positioned in a manner to sterilize gaming pieces 34. UV light source(s) 50 may be positioned above gaming pieces 34, below the pieces, to the side of the pieces, or combination thereof. Each UV light source 50 may be positioned a certain distance away from gaming pieces 34 to sterilize the pieces without damage (e.g., as described in the examples below). For example, each UV light source 50 may be positioned at a distance from gaming pieces 34 that is greater than, equal to, or between any two of: 0.5, 1.0, 1.5, 2.0, 2.5, 3.0, 3.5, 4.0, 4.5, 5.0, 5.5, 6.0, 6.5, 7.0, 7.5, 8.0, 9.0, 10.0, 11.0, or 12.0 inches. In some embodiments, at least one UV light source 50 may be moveable (e.g., via actuators) to adjust the distance between the UV light source and the gaming pieces. In this way, the position of the UV light sources 50 may be adjusted to change the energy applied to gaming devices 34.

In a non-limiting example, device 10d may, but need not, include a platform 18d that is configured to support gaming pieces 34 while they are disposed within chamber 16d. As shown in FIG. 6B, platform 18d may be spaced from the bottom or top surfaces of housing 14d so that the platform is vertically disposed between two UV light sources 50. In some embodiments, one or more additional UV light sources 50 may be aligned horizontally with gaming pieces 34 to sterilize multiple sides of the gaming pieces without having to move the pieces. Platform 18d may include any material that, at least partially, allows UV light to pass through the material. For example, platform 18d may include a suitable plastic, glass (e.g., fused quartz), or the like. While platform 18d is shown being positioned between two UV light sources 50, the platform may be positioned between a UV light source 50 and a reflector (e.g., one or more mirrors) so light emitted from the UV light source may be reflected back towards the platform and gaming pieces 34. In this way and others, device 10d may be configured to sterilize all faces of gaming pieces 34 without having to rotate the gaming pieces.

Device 10d may include one or more other components that are not shown. For example, device 10d may include one or more other components described in devices 10, 10a, 10b, 10c, above, such as a feeder (e.g., 30), sorting system (e.g., 46), shuffling system (e.g., 42), dispenser (e.g., 38), or combination thereof. Additionally, or alternatively, device 10d may include one or more sensors and/or timers 54, such as, for example, a UV sensor, temperature sensor (e.g., thermometer), timer, mirrors, reflectors, imaging devices (e.g., camera, UV camera, thermal imaging camera, or the like). In some embodiments, device 10d includes a controller 60 configured to control the one or more components of the device. For example, controller 60 may be able to initiate operation(s) of UV light source 50, one or more sensors and/or timers 54, or other components of devices (10, 10a, 10b, 10c, 10d), to perform any of the functions as described herein.

Controller 60 may be physically or wirelessly coupled to one or more of the other components of device 10d and configured to control operation of the system via one or more user-initiated or automatic commands or parameters. In some embodiments, controller 60 can include a processor (e.g., a microcontroller/microprocessor, a central processing unit (CPU), a field-programmable gate array (FPGA) device, an application-specific integrated circuits (ASIC), another hardware device, a firmware device, or any combination thereof) and a memory (e.g., a computer-readable storage device) configured to store instructions, one or more thresholds, and one or more data sets, or the like. In some embodiments, controller 60 may include, or be coupled to, a transceiver, one or more interface(s), one or more I/O device(s), a power source, one or more sensor(s), other signaling device, or combination thereof.

In some embodiments, the one or more operations initiated or performed by controller 60 include identifying a gaming piece 34. In some such embodiments, such identification may be achieved via an imaging device (e.g., 54), sorting system (e.g., 46), scale, an input (e.g., user input), or the like. In some embodiments, the one or more operations initiated or performed by controller 60 may be based on the identification or determination of the type of gaming piece 34. In an illustrative example, controller 60 may store (e.g., via memory) sanitation profiles associated with each respective gaming pieces of a plurality of gaming pieces. The sanitation profiles can include a target distance, temperature threshold, risk level, target SAL, target log reduction, FO-values, decimal reduction values (D-values), Z-values, combination thereof, or the like.

In some embodiments, the one or more operations initiated or performed by controller 60 include activating the one or more UV light source(s) 50. For example, controller 60 may activate UV light source(s) 50 for a specific duration (e.g., continuously for more than 5, 10, 15, 20, 25, 30, 40, 50, 60, 70, 80, 90, 100, 125, 150, 175, or 200 seconds), in a specific pattern (e.g., setting or changing a duty cycle, pulse duration, pulse rate, and number of pulses), at a frequency range (e.g., UV-A, UV-B, UV-C, or other light having a wavelength between 180 and 800 nanometers), at an energy range (e.g., 1 to 800 Joules per pulse). In a more specific example, controller 60 may activate UV light source(s) 50 to emit between 2 and 150 UV pulses (e.g., equal to or between any two of 3, 5, 10, 15, or 25 pulses).

Controller 60 may control the intensity (e.g., energy, wavelength, pulse rate) based on the operation of the device (e.g., 10). As an illustrative example, controller 60 or UV light source(s) can be configured to emit light at an energy between 0.5 and 600 joules per pulse (e.g., greater than, equal to, or between any two of 5, 10, 15, 20, 25, 30, 40, 50, 60, 75, 100, 120, 140, 160, 180, 200, 225, 250, 275, 300, 350, 400, 450, or 500 joules per pulse). To further illustrate, in embodiments in which the device (e.g., 10) is configured to store gaming pieces 34, controller 60 may activate UV light source(s) 50 to emit light at a lower energy for a longer duration. On the other hand, in embodiments in which the device (e.g., 10) is configured to quickly receive and dispense gaming pieces 34, controller 60 may activate UV light source(s) 50 to emit light at a higher energy for a shorter duration. For example, UV light source(s) 50 within sorting devices (e.g., card shufflers or chip sorters) may be configured to emit pulsed UV light at a high enough energy to adequately sterilize gaming pieces 34 during the normal sorting operation (e.g., within just a few minutes or even in seconds). In this way and others, the present devices may be employed without adding delays or stopping game play.

In some embodiments, controller 60 can activate UV light source(s) 50 based on one or more inputs or instructions (e.g., programmable control logic). For example, a characteristic (e.g., total duration, duty cycle, wavelength, pulse duration, number of pulses, energy, or the like) of at least one UV light source(s) 50 may be altered based on a user input, sanitation profiles of gaming piece 34, or other determination. In a specific non-limiting example, controller 60 may determine a type of gaming pieces 34 within housing 14 and, based on the type of gaming piece, select a number of pulses to be emitted from UV light source. In other embodiments, controller 60 may select the number of pulses emitted from UV light source 50 (or any other characteristic) based on sanitation profiles or other features associated with gaming pieces 34 such as, for example, color, weight, material, shape, or the like. In another specific non-limiting example, controller 60 may activate UV light source(s) 50 until gaming pieces reach a temperature threshold (e.g., detected by a temperature sensor). In such embodiments, the temperature threshold may correspond to a temperature at which the gaming piece begins to experience deterioration (e.g., burning, color change, chemical change, or the like). Additionally, or alternatively, controller 60 may control UV light source(s) 50 to maintain gaming pieces 34 at a target temperature or temperature range (e.g., greater than, equal to, or between any two of: 85, 90, 95, 100, 105, 110, 115, 120, 125, or 130° F.). In some embodiments, controller 60 or UV light source(s) 50 are configured to otherwise limit UV exposure to gaming pieces 34 (e.g., by controlling operation distance, pulse rate, pulse energy, duration, number of pulses, or the like). In this way, and others, the devices (e.g., 10) may be configured to sterilize gaming pieces 34 without damage or deterioration to the gaming pieces.

In some embodiments, the one or more operations initiated or performed by controller 60 include moving the one or more UV light source(s) 50 relative to the gaming pieces. For example, controller 60 may determine a type of gaming pieces 34 within housing 14 and, based on the type of gamine piece, actuate at least one UV light source(s) 50 to a target distance. In some embodiments, the target distance may be measured from the UV light source 50 to a closest surface of gaming pieces 34 and may be greater than, equal to, or between any two of: 0.5, 1.0, 1.5, 2.0, 2.5, 3.0, 3.5, 4.0, 4.5, 5.0, 5.5, 6.0, 6.5, 7.0, 7.5, 8.0, 9.0, 10.0, 11.0, or 12.0 inches. In some embodiments, controller 60 (e.g., via memory) may store a respective target distance for each type of gamine piece (e.g., 34). In this way and others, UV light source 50 may be positioned at a distance from gaming pieces 34 to prevent deterioration of the pieces.

### Examples

The present invention will be described in greater detail by way of specific examples. The following examples are offered for illustrative purposes only and are not intended to limit the present invention in any manner. Those skilled in the art will readily recognize a variety of non-critical parameters that can be changed or modified to yield essentially the same results.

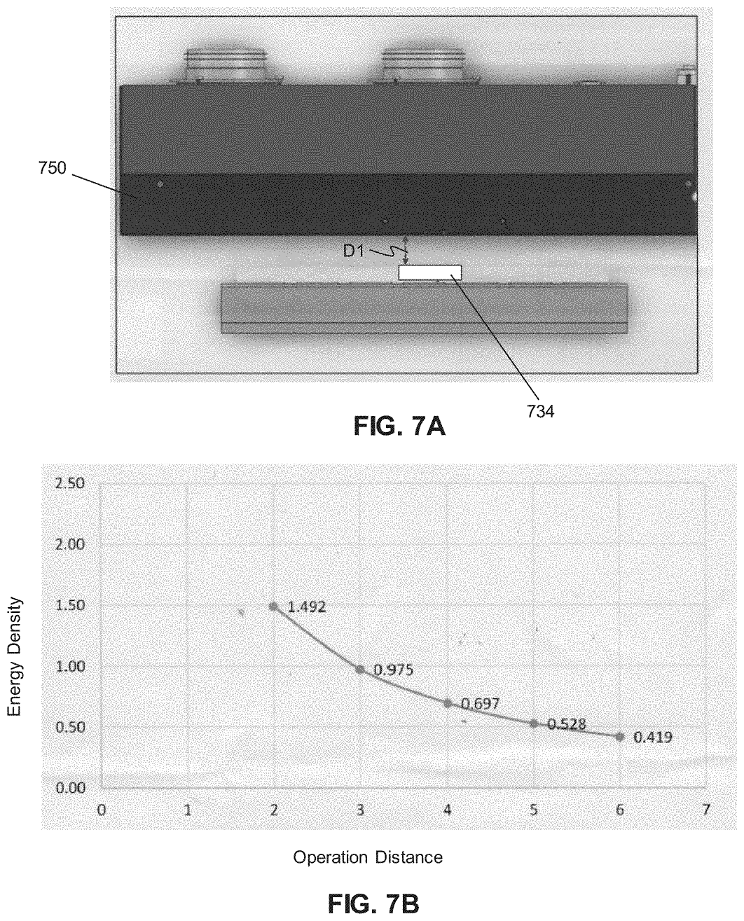

Sanitation tests were performed on three types of sample gaming pieces: roulette chips, poker chips, and gaming die. For each test a 16 inch linear lamp from XENON (RC 800) was used as the UV light source and operated to emit UV light pulses at 505 Joules per pulse (J/p). An example of the experimental setup is shown in FIG. 7A. During the tests, an operation distance D1—the distance between the UV light source 750 and the closest surface of the sample gaming piece 734—and the number of pulses were varied, as described below. The standard energy density of UV light source 750 was measured at operation distances (e.g., D1) between two inches and six inches. The resulting energy densities are depicted in FIG. 7B, with the highest energy density being 1.492 J/p/cm² at 2 inches from UV light source 750 and the lowest energy density being 0.419 J/p/cm² at 6 inches from the UV light source.

### Roulette Chips

Blue, brown, and red roulette chips (e.g., 734) were placed at an operational distance of 2 inches from UV light source 750 and subjected to 5, 10, or 40 pulses on each side of the chip. Each of chips were maintained at a constant temperature of 68° F. before being subjected to the UV light source 750. Each chip was individually placed 2 inches from UV light source 750 and the specified number of pulses were emitted toward the first side of the chip. After this first set of pulses were emitted, the temperature of the chip was recorded using an infrared gun. The chip was then flipped over, a second set of the same number of pulses were emitted toward the second side of the chip, and the temperature was recorded. Table 1, reproduced below, shows the resulting temperatures.

TABLE 1

| | | Pulses | Temperature after | Temperature after |
|---|---|---|---|---|
| Sample | | (each | pulsing first side | pulsing second side |
| Number | Color | side) | (° F.) | (° F.) |
| 1 | Blue | 5 | 84.8 | 86.2 |
| 2 | Blue | 5 | 73.5 | 86.5 |
| 3 | Blue | 5 | 77.9 | 89.5 |
| 4 | Blue | 10 | 89.3 | 96.9 |
| 5 | Blue | 10 | 88.8 | 116 |
| 6 | Blue | 10 | 90.6 | 117 |
| 7 | Brown | 5 | 88.8 | 95 |
| 8 | Brown | 5 | 89.4 | 95.4 |
| 9 | Brown | 5 | 87.4 | 89.9 |
| 10 | Brown | 10 | 83.4 | 109 |
| 11 | Brown | 10 | 89.4 | 115 |
| 12 | Brown | 10 | 86.4 | 111 |
| 13 | Red | 5 | 86.2 | 93.4 |
| 14 | Red | 5 | 85.6 | 100.2 |
| 15 | Red | 5 | 77.6 | 93.8 |
| 16 | Red | 10 | 102 | 109.2 |
| 17 | Red | 10 | 82.6 | 114 |
| 18 | Red | 10 | 90.4 | 102 |
| 19 | Blue | 40 | 103 | 121 |
| 20 | Brown | 40 | 138 | 132 |
| 21 | Red | 40 | 132 | 118 |

After the final temperature was recorded, each chip was visually inspected for changes. While no visual changes were found, each of the chips subjected to 40 pulses per side had a distinct smell—similar to that of burning plastic. As shown in TABLE 1, for the chips subjected to 5 or 10 pulses on each side, the temperatures of the chips increased directly with the number of pulses. For the chips subjected to 5 pulses per side, the average temperature of the chips was 83.46° F. after the first set of pulses and 89.21° F. after the second set of pulses. For the chips subjected to 10 pulses per side, the average temperature of the chips was 92.21° F. after the first set of pulses and 110.01° F. after the second set of pulses. No burning smell was detected during treatment of the chips using 10 pulses per side. For two of the three chips subjected to 40 pulses, the temperature of the chips actually decreased during delivery of the pulses to the second side of the chip. Accordingly, at a certain point (e.g., around 120-140° F.), the chips seemed to reach a maximum temperature independent of any additional pulses. While no visual damage was seen, the temperature of the chips should be kept below 120° F., preferably below 115 or 110° F., during UV treatment to prevent unpleasant odors.

The color of the chip may have some effect on the temperature, although a larger sample is needed to verify this relationship.

### Poker Chips

Five differently colored poker chips were treated using the UV light source to detect deterioration of the chips. Each chip was subjected to 5 pulses per side and any changes to the physical properties of the chip were recorded. The chips were tested at operation distances of 2 inches and 4 inches, the results are illustrated in TABLE 2, below.

TABLE 2

| Sample | | Distance | Pulses | |
|---|---|---|---|---|
| Number | Color | (inches) | (each side) | Notable Changes |
| 1 | Purple | 2 | 5 | Color changed significantly, |
| | | | | burn smell |
| 2 | Purple | 4 | 5 | No changes |
| 3 | Black | 2 | 2 | Color changed, burn smell |
| 4 | Black | 4 | 5 | No changes |
| 5 | Red | 2 | 5 | Color changed, burn smell |
| 6 | Red | 4 | 5 | No changes |
| 7 | Green | 2 | 5 | Color changed, burn smell |
| 8 | Green | 4 | 5 | No changes |
| 9 | White | 2 | 3 | Color changed slightly, burn |
| | | | | smell |
| 10 | White | 4 | 5 | No changes |

As shown in TABLE 2, color of the chips has little effect on the deterioration of the poker chips. Unlike roulette chips, described above, 5 pulses per side at a distance of 2 inches had a noticeable effect on the poker chips and produced the same smell as the 40 pulse roulette chips. Even with only 2 pulses per side at 2 inches, as seen in the black poker chip, the color of the chip was changed and an unpleasant smell was produced. However, when the operation distance (e.g., D1) was changed to 4 inches, there were no noticeable color changes for any of the colored poker chips. Additionally, no noticeable smell was produced at an operation distance of 4 inches. Accordingly, the operation distance should be maintained above a certain distance threshold (e.g., 2 or 3 inches for poker chips) to prevent deterioration.

### Die

Three six-sided, cubed die were treated using UV light source 750 to detect deterioration and UV exposure for the different sides of the dice. A color-changing, UV intensity label was attached to each side of the die. The UV intensity label initially is a yellow color and as the label is exposed to UV light, it becomes green. After continued UV exposure, the label transitions to a deeper shade of green and eventually reaches solid blue at maximum UV exposure. Each dice was subjected to a first set of 5 pulses on a first side of the dice. The dice was then flipped 180 degrees so that the first side faced away from UV light source 750 subjected to a second set of 5 more pulses on a second side that is opposite the first side. The die were tested at operation distances of 2 inches and 4 inches, the results are illustrated in TABLE 3, below.

TABLE 3

| | | Distance | Pulses | |
|---|---|---|---|---|
| Number | Type | (inches) | (each side) | Notable Changes |
| 1 | Dice | 2 | 5 | Slightly burned |
| 2 | Dice | 4 | 5 | No changes |
| 3 | Dice | 4 | 5 | No changes |

As shown in TABLE 3, the dice positioned at an operation distance of 2 inches was slightly burned, while the die positioned at a 4 inch operation distance had no noticeable changes. Similar to the poker chips, UV light source 750 should be positioned greater than 2 inches from the die. Alternatively, a lower pulse energy may be

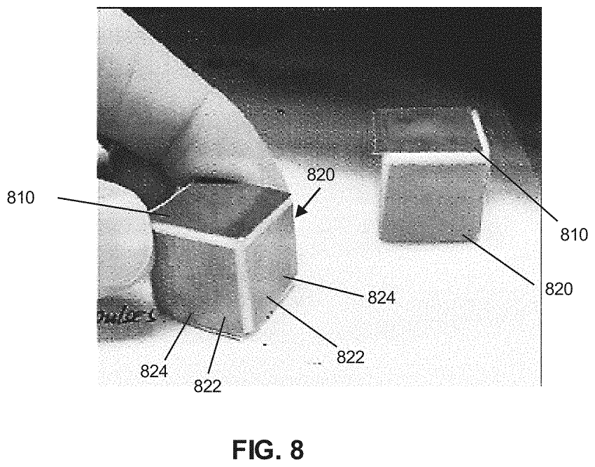

The UV intensity labels indicated maximum exposure had been reached for each side of the die after the rotation. However, after the first set of pulses the UV exposure was different between the first (top) surface, the side surfaces, and the second (bottom) surface. FIG. 8 shows the differences of UV exposure for two of the die after the first set of pulses, but before the second set of pulses. Each illustrated dice includes a top UV indicator 810 placed on the first surface and a side UV indicator 820 placed on the side surfaces. As shown, the top UV indicator 810 is much darker than the side UV indicators 820. Further, the top UV indicator 810 is evenly colored indicating evenly distributed UV exposure. On the other hand, some of the side UV indicators 820 have lighter areas 822 that had less UV exposure than adjacent darker areas 824. Additionally, a bottom UV indicator positioned on the second (bottom) surface displayed very little UV exposure. Accordingly, for uniform exposure, the gaming pieces may need to be rotated during treatment. Additionally, or alternatively, additional UV light sources may be utilized and positioned opposite one another for better UV coverage. In yet more embodiments, a reflector may positioned opposite UV light source 750 to reflect the emitted light back toward the UV light source.

The above specification and examples provide a complete description of the structure and use of illustrative configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the methods and systems are not intended to be limited to the particular forms disclosed. Rather, they include all modifications and alternatives falling within the scope of the claims, and configurations other than the one shown may include some or all of the features of the depicted configurations. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one configuration or may relate to several configurations. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. A sterilizing gaming device comprising:
a housing defining an interior chamber configured to receive a plurality of gaming pieces;
a light source coupled to the housing and configured to emit ultraviolet (UV) light;
wherein, when the plurality of gaming pieces are disposed within the chamber the light source is configured to direct the emitted UV light toward the gaming pieces at an energy sufficient to sterilize the gaming pieces.

2. The sterilizing gaming device of claim 1, further comprising a sorting system configured to sort the plurality of gaming pieces.

3. The sterilizing gaming device of claim 2, wherein the sterilizing gaming device corresponds to a card shuffler, a chip sorter, or a cash drop box.

4. The sterilizing gaming device of claim 1, wherein the light source is configured to emit pulsed UV light.

5. The sterilizing gaming device of claim 1, further comprising a controller in communication with the light source, the controller configured to:
identify a type of the plurality of gaming pieces; and
based on the type, select a number of pulses to be delivered to the plurality of gaming pieces.

6. The sterilizing gaming device of claim 1, wherein:
the housing includes a platform disposed within the chamber;
the light source is disposed on a first side of the platform; and
one other light source or a reflector is disposed on a second side of the platform, the second side being opposite the first side.

7. The sterilizing gaming device of claim 1, wherein the housing defines a first opening configured to receive the gaming pieces and a second opening configured to dispense the gaming pieces.

8. The sterilizing gaming device of claim 7, wherein the light source is positioned such that the gaming pieces are sterilized as they pass through the first opening or the second opening.

9. The sterilizing gaming device of claim 1, wherein the housing is coupled to a card playing table.

10. A sterilizing gaming device comprising:
a housing configured to be coupled to gaming equipment, the housing having an opening configured to transfer gaming pieces between the housing and the gaming equipment;
one or more ultraviolet (UV) light sources coupled to the housing and positioned to deliver UV light to the gaming pieces.

11. The sterilizing gaming device of claim 10, wherein at least one of the one or more ultraviolet (UV) light sources are positioned within the housing.

12. The sterilizing gaming device of claim 10, wherein the gaming equipment corresponds to a card shuffler or a chip sorter.

13. The sterilizing gaming device of claim 10, wherein the one or more ultraviolet (UV) light sources are configured to emit pulsed UV light such that the gaming pieces are subject to at least 2 log reduction.

14. The sterilizing gaming device of claim 10, wherein the housing is coupled to the gaming equipment such that the gaming pieces are received by the housing from the gaming equipment.

15. The sterilizing gaming device of claim 10, wherein the housing is coupled to the gaming equipment such that the gaming pieces are delivered to the gaming equipment from the housing.

16. The sterilizing gaming device of claim 10, wherein the gaming pieces include chips, die, cards, or cash.

17. A method of sanitizing gaming pieces, the method comprising:
applying ultraviolet (UV) light, via a gaming device having one or more UV light sources, to a plurality of gaming pieces to sterilize the gaming pieces;
wherein the gaming device is coupled to a card playing table.

18. The method of claim 17, wherein applying ultraviolet light comprises applying five or more light pulses, each light pulse having an energy greater than 12 joules per pulse.

19. The method of claim 17, wherein the UV light includes a wavelength that is between 100 and 400 nanometers.

20. The method of claim 17, further comprising:
disposing the gaming pieces within a housing of the gaming device;
sorting the gaming pieces; and
after the gaming pieces are sorted and sterilized, dispensing the gaming pieces from the housing.
